(19) United States
(12) Patent Application Publication
(10) Pub. No.: US 2012/0065566 A1
(43) Pub. Date: Mar. 15, 2012

(54) METHOD AND APPRATUS FOR RELEASABLY RETAINING BANDAGES

(75) Inventor: Bernard Bar-Natan, Givatayim (IL)

(73) Assignee: FIRST CARE PRODUCTS LTD., Lod (IL)

(21) Appl. No.: 13/260,102

(22) PCT Filed: Mar. 22, 2010
(86) PCT No.: PCT/IL2010/000235

§ 371 Date: Nov. 8, 2011

(30) Foreign Application Priority Data
Mar. 26, 2009 (IL) 197854

(51) Int. Cl. A61F 13/00; A61F 15/00

(52) U.S. Cl. 602/53; 493/394; 602/42

(57) ABSTRACT

There is provided herein a bandage folded upon itself to have a plurality of panels or rolled and a device having an elongated body portion and end elements at its two opposite ends sized and shaped to extend through several panels of the folded bandage, and retain the bandage in a folded configuration, while allowing a user to unfold the bandage without removing the device. The bandage may be unfolded, panel-by-panel, by pulling on an end panel of the bandage, causing it to be released from the device, and pulling on released panels or topmost retained panels to free the topmost retained panels, each in turn.

### FIELD

The invention relates to bandages, such as pressure bandages, and devices for use therewith.

### BACKGROUND

A pressure (or compression) bandage may be in the form of an elongate strip (or “web”) of material (such as cloth, gauze or other material) for wrapping an injured part of a patient's body, such as the patient's arm, leg, head, neck or torso.

The purpose of bandages, in addition to protecting exposed wounds is to prevent loss of blood by applying pressure against the bleeding wound. Bandages may be packaged to be carried and used for example, by military personnel, police, ambulances, hikers and campers, and may also be part of first-aid kits, for use in clinics, hospitals, factories, offices and the home.

As described in commonly-owned U.S. Pat. No. 5,628,723, bandages include a dressing which includes one or more soft, absorbent portions, typically made of cotton, sandwiched between a sterile non-adherent pad and a structural layer. The pad is adapted to be placed on an open wound. The bandages also include a pressure enhancement member disposed on a non-wound side of the bandage, opposite the dressing. When the bandage is placed on the wounded body part, the pressure enhancement member should be guided to cover the wound such that a user (person applying the bandage, for example, any caregiver such as a medic or a doctor or even an unqualified or untrained person, such as the wounded person himself or anyone else, can apply local pressure to the wound. The subsequent wrappings of the web portion on the pressure enhancement member increases the local pressure applied to the wound.

Prior to packaging for sale and distribution, bandages are generally arranged in such way that the web of the bandage (either with or without integral dressing) is rolled up and packaged for sale and distribution. One of the multiple problems when using such bandages is that when applying the bandage to a wound, as the user starts to unroll the web, it may unroll entirely and drop to the ground. There also exist self-adhesive bandages (that use for example a hook-and-loop fasteners such as Velcro) but these may provoke an allergic reaction and may not work in wet environment.

### SUMMARY

This summary section of the patent application is intended to provide an overview of the subject matter disclosed herein, in a form lengthier than an “abstract”, and should not be construed as limiting the invention to any features described in this summary section.

Some embodiments of the invention are generally directed to a device adapted in use for maintaining a web of a bandage in a rolled or folded configuration, while allowing the user (person applying the bandage, caregiver and/or victim) to easily and gradually unroll or unfold the web, sequentially, such as step-by-step, as needed, without the remaining web unrolling or unfolding itself and dropping to the ground. The device may be referred to as a “fold-retaining” or “stop+go”) device.

According to an embodiment of the invention, there is provided a bandage comprising: an elongated web adapted to be folded upon itself, the web, when being in a folded configuration, comprises a plurality of panels abutting one another; and a fold-retaining device comprising an elongated body portion, a top end element and a bottom end element opposing to the top end element, wherein the device is adapted to extend through two or more of the plurality of panels and releasably retain the web in a folded configuration. The fold-retaining device may be adapted to allow a user to gradually unfold the web without removing the device. The fold-retaining device may be adapted to allow a user to unfold one or more panels by slipping the one or more panels, panel-by-panel, over the top end element of the fold-retaining device, while maintaining other panels folded. The device may be sufficiently long to extend through the folded web, and is sufficiently thin to pierce through a material of the web of the bandage. A length (L) of the body portion of the fold-retaining device may be approximately equal to, or slightly greater than, or slightly less than a thickness (T) of the folded web. The web may be zig-zag folded. The bandage may further comprise a dressing adapted to cover a wound, the dressing is disposed on a surface of a selected one of the panels. The bandage may further comprise a pressure enhancement member disposed on an opposite surface of the selected one of the panels, proximal to the dressing. At least one of the top end element and the bottom end element may have a thickness (D2), which is greater than a thickness (D1) of the body portion. The body portion may be essentially round in cross-section, and the thickness (D1) is a diameter. The body portion may comprise one or more materials selected from the group consisting of polymers, synthetic polymers (such as nylon and plastic, for example types of plastic that are approved for medical use), silicon, and cotton. The body portion may comprise a thread, which comprises a textile material comprising natural fibers, synthetic fibers or a combination thereof The body portion may comprise a thread and the end elements may be formed of a rigid material. The top end element may be adapted to be disposed exterior a first of the panels, and the bottom end element is adapted to be disposed exterior a last of the folded panels. At least one of the top and bottom end elements may be essentially ball-shaped. At least one of the top and bottom end elements may be Y-shaped. A Y-shaped end element, when collapsed, may have a width dimension (W1) which is substantially equal to a thickness (D1) of the body portion; and the Y-shaped end element, when spread, may have a width dimension (W2) which is greater than the thickness (D1) of the body portion. The top and bottom end elements may be substantially the same as one another. The top and bottom end elements may be different from one another. At least one of the top and bottom end elements may be integrally formed with the body portion. At least one of the top and bottom end elements may be formed separately from the body portion, and are mounted thereto. At least one of the top and bottom end elements may comprise two or more elongated fingers extending in a spread position from the body portion, wherein the two or more elongated fingers are adapted to collapse when one or more panels are being pulled to unfold the web.

According to an embodiment of the invention, there is provided a fold-retaining device comprising: an elongated body portion, a top end element and a bottom end element opposing to the top end element, wherein the device is adapted to extend through two or more panels of an elongated folded web of a bandage, and releasably retain the web in a folded configuration. The device may be further adapted to allow a user of the bandage to gradually unfold the web without removing the device. The device may be further adapted to allow a user of the bandage to unfold one or more panels by slipping the one or more panels, panel-by-panel, over the top end element of the fold-retaining device, while maintaining other panels folded sufficiently long to extend through the folded web, and is sufficiently thin to pierce through a material of the web of the bandage. A length (L) of the body portion of the fold-retaining device may be approximately equal to, or greater than, or less than a thickness (T) of the folded web. The web of the bandage may be zig-zag folded. At least one of the top end element and the bottom end element may have a thickness (D2), which is greater than a thickness (D1) of the body portion. The body portion may be essentially round in cross-section, and the thickness (D1) may be a diameter. The body portion may comprise one or more materials selected from the group consisting of polymers, synthetic polymers (such as nylon and plastic, for example types of plastic that are approved for medical use), silicon, and cotton. The body portion may comprise a thread, which may comprise a textile material comprising natural fibers, synthetic fibers or a combination thereof The main body portion may comprise a thread and the end elements may be formed of a rigid material. The top end element may be adapted to be disposed exterior a first of the panels, and the bottom end element is adapted to be disposed exterior a last of the folded panels. At least one of the top and bottom end elements may be essentially ball-shaped. At least one of the top and bottom end elements may be Y-shaped. The Y-shaped end element, when collapsed, may have a width dimension (W1) which is substantially equal to a thickness (D1) of the body portion; and the Y-shaped end element, when spread, may have a width dimension (W2) which is greater than the thickness (D1) of the body portion. The top and bottom end elements may be substantially the same as one another. The top and bottom end elements may be different from one another. At least one of the top and bottom end elements may be integrally formed with the body portion. At least one of the top and bottom end elements may be formed separately from the body portion, and are mounted thereto. At least one of the top and bottom end elements may comprise two or more elongated fingers extending in a spread position from the body portion, wherein the two or more elongated fingers are adapted to collapse when one or more panels are being pulled to unfold the web.

According to an embodiment of the invention, there is provided a method of releasably retaining an elongated web of a bandage in a folded configuration, the method may comprise: folding an elongated web of a bandage to form a plurality of panels abutting one another; and inserting a fold-retaining device through two or more of the plurality of panels such that the fold-retaining device is releasably retaining the web in a folded configuration. The method may further include prior to inserting the fold-retaining device through the two or more of the plurality of panels, first compressing the folded bandage.

According to an embodiment of the invention, there is provided a method of releasably retaining an elongated web of a bandage in a folded configuration, the method may comprise: folding an elongated web of a bandage to form a plurality of panels abutting one another; and inserting a heated element through two or more of the plurality of folds, wherein fibers of one fold that are in a direct contact with the heated element are melted and stick to fibers of one or more adjacent folds in the area of the path of the heated element, thereby releasably retaining the web in a folded configuration. The method may further include compressing the folded bandage, prior to inserting the heated element.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures (FIGs.) are listed below.

FIG. 1A schematically shows, in cross-sectional view, a bandage.

FIG. 1B schematically shows, in cross-sectional view, a bandage.

FIG. 2 schematically shows, in cross-sectional view, an embodiment of the device of the present invention.

FIG. 2A schematically shows, in cross-sectional view, a bandage with an embodiment of the device of the present invention.

FIG. 2B schematically shows, in cross-sectional view, a bandage with an embodiment of the device of the present invention.

FIG. 2C schematically shows, in cross-sectional view, a bandage with an embodiment of the device of the present invention.

FIG. 2D schematically shows, in cross-sectional view, an embodiment of the device of the present invention.

FIG. 2E schematically shows, in cross-sectional view, an embodiment of the device of the present invention.

FIG. 3A schematically shows, in cross-sectional view, an embodiment of the device of the present invention.

FIG. 3B schematically shows, in cross-sectional view, an embodiment of the device of the present invention.

FIG. 3C schematically shows, in cross-sectional view, a bandage with an embodiment of the device of the present invention.

FIG. 3D schematically shows, in cross-sectional view, an embodiment of the device of the present invention.

FIG. 4 schematically shows, in cross-sectional view, a bandage with an embodiment of the device of the present invention.

### DETAILED DESCRIPTION OF EMBODIMENTS

In the following description, various aspects of techniques for releasably retaining a bandage comprising an elongated web which is folded upon itself will be described. For the purpose of explanation, specific configurations and details are set forth in order to provide a thorough understanding of the techniques. However, it will also be apparent to one skilled in the art that the techniques may be practiced without specific details being presented herein. Furthermore, well-known features may be omitted or simplified in order not to obscure the description(s) of the techniques.

After manufacture, bandages (either with or without integral dressing) may be rolled up and packaged for sale and distribution. An alternative to rolling up the bandage is to fold it. Folding a material once (n=1) will result in there being two (n+1) “panels”, each of which may (or may not) be the same size as each other. The resulting panels are generally parallel with one another, and substantially abutting one another.

A material may folded with “zig-zag” folds, will have two or more (n>=2) parallel folds, each folding the material in opposite directions, resulting in a number (n+1) of panels, each of which may (or may not) be the same size as each other. The resulting panels are generally parallel with one another, and substantially abutting one another. Seen from above, zig-zag folds resemble a Z or M. Zig-zag folds may also be referred to as “accordion folds” or “z folds”. A typical pressure bandage may be zig zag folded, with several (such as n=5−10) folds, resulting in several (n+1) panels.

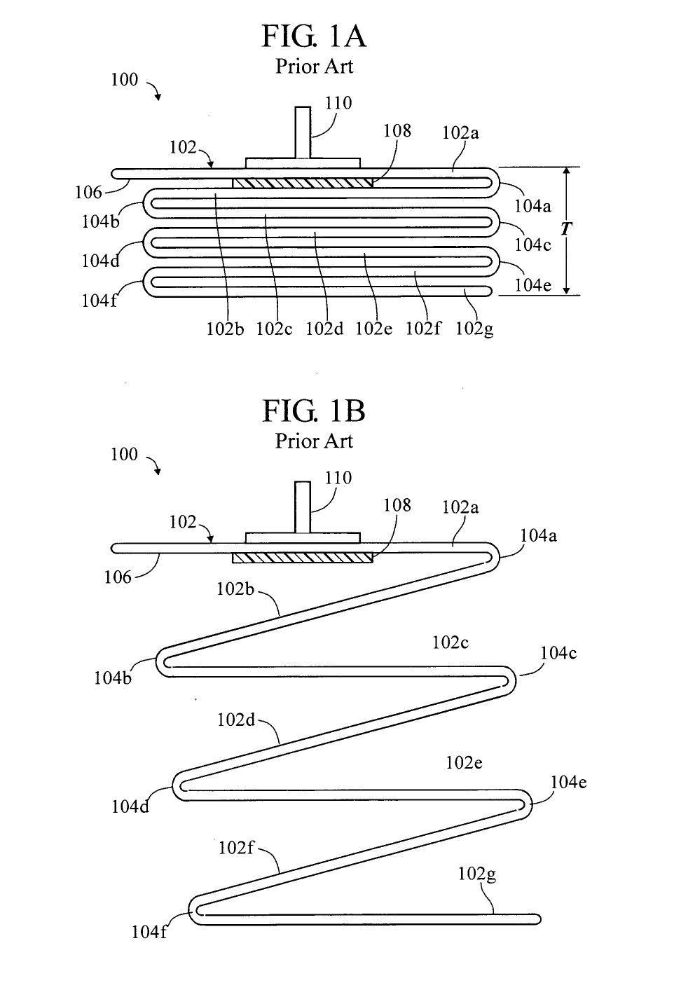

FIG. 1A illustrates a bandage 100, generally comprising an elongated web 102. The web 102 is folded upon itself, such as zig-zag folded, with six (n=6) folds 104a-104f, resulting in seven (n+1) panels 102a-102g. Of course, n can represent any number (such as 3, 4, 5, 6, 7, 8. . . n), 6 being just an example.

The panel 102a is an “end panel”. A dressing 108 may be disposed on a surface of the end panel 102a. A pressure enhancement member 110, such as described in commonly-owned U.S. Pat. No. 5,628,723, may be disposed on a surface of the end panel 102a opposite the dressing 108.

It may be noted that the end panel 102a has been drawn slightly longer than the rest of the panels 102b-g. This is indicative of an “additional” web portion 106, which typically extends about 5 cm from the dressing 108 and which may be grasped by a user when applying the bandage 100 on a wound (not shown).

In FIG. 1A, the bandage 100 is shown in its folded configuration, neatly folded, with the panels 102a-102g generally parallel with one another, and substantially abutting one another. For example, the panel 102a abuts the panel 102b, the panel 102b abuts the panels 102a and 102c, the panel 102c abuts the panels 102b and 102d, the panel 102d abuts the panels 102c and 102e, the panel 102e abuts the panels 102c and 102f, and the panel 102f abuts the panels 102e and 102g.

Typical dimensions for a pressure bandage (namely, the web) may be 10-15 cm wide, and at least one meter long. Typical dimensions for the dressing 108 may be 10-15 cm square. The web material may be approximately 0.5-1.5 mm thick. When folded-up, the bandage 100 has an overall thickness “T”, exclusive of the pressure enhancement member 110. The thickness “T” may, for example, be approximately 1-3 cm (10-30 mm). In this example, the overall thickness “T” is approximately equal to seven times the thickness of each panel (102a-102f), plus the thickness of the dressing 108, taking into consideration that the web and dressing are formed of “soft” materials (such as cotton, gauze, elasticized fabric, and the like) which may be compressed when packaged. In addition, by compressing during packing, air can be removed and the thickness “T” can be reduced.

When the bandage 100 is removed from its package (not shown), the user (person applying the bandage) may hold (grasp) it in their hand(s), thereby maintaining the bandage 100 in its zig-zag folded configuration. But as the first few panels (such as 102a and 102b) are unfolded by the user, for applying the bandage to a wound (such as wrapping around a victim's limb), the remaining panels (such as 102c-102g) may be dropped, unfold and extend (fall) to the to the ground. This is generally undesirable since not only can the bandage become contaminated, but it can also become unwieldy, or snagged on something. This “unfolded” condition of the bandage 100 is illustrated in FIG. 1B. It should be understood that, when unfolded, there may not be these small (such as 15 degree) angles between successive panels, but rather the entire bandage 100 would tend to straighten out until it hit the ground.

There will now be described embodiments of method and apparatus for maintaining the panels of a folded, such as a zig-zag folded bandage abutting one another, while allowing the person applying the bandage (user, caregiver) to easily unfold the panels, sequentially, such as one-by-one (panel-by-panel), as needed (or un-roll, step-by-step), without the remaining panels (rolls) unfolding themselves and dropping to the ground. The devices described herein may be referred to herein as “fold-retaining” devices. The feature of retaining the bandage in folded condition, then allowing it to be unfolded by the user may be referred to herein as “releasably retaining”.

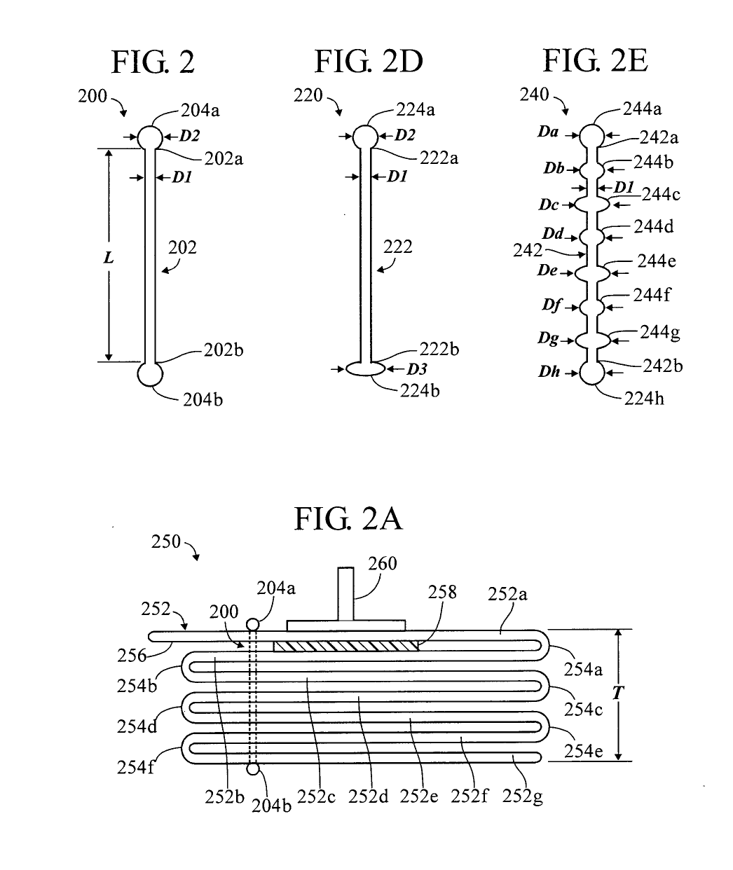

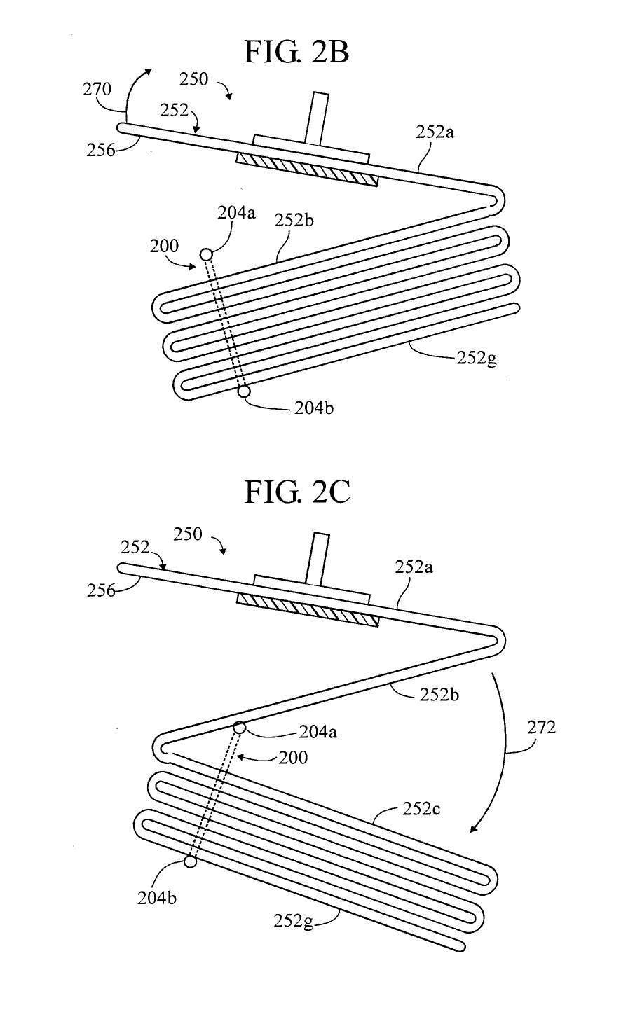

FIG. 2 shows an embodiment of a fold-retaining device 200. The device 200 is an elongated member, such as in the form of a pin. The device 200 has an elongated main body portion 202 with two opposite ends 202a and 202b. The body portion 202 may be round in cross-section. The body portion 202 has a length “L” and a thickness or diameter (if round in cross-section) “D1”. For purposes of this description, the end 202a is a “top” end, and the end 202b is a “bottom end”.

The body portion 202 may be formed of a structural plastic material. Or, it may be formed as a thread, from a textile material such as cotton, or other natural or synthetic fibers. Other materials, such as, silicon, polyethylene, or any other appropriate materials can also be used.

End elements 204a and 204b having increased thickness or diameter (if round) “D2” may be disposed at the opposite ends 202a and 202b, respectively, of the body portion 202 of the device 200. The dimension “D2” is greater than the dimension D1”. For purposes of this description, the end element 204a is a “top” end element, and the end element 204b is a “bottom” end element. The top and bottom end elements 202a and 202b may be ball-shaped (round).

The top and bottom end elements 204a and 204b may be formed integrally with the body portion 202, or separately therefrom and appended (mounted) thereto. (One end element may be formed integrally and the other may be formed separately.) For a body portion 202 which is a thread, the end elements may be balls formed of a rigid material, such as plastic. (The thread with rigid ends may be likened to shoelaces, tipped with “aglets”. However, the aglets are typically of a lesser diameter than the lace, to assist in threading the shoelace through eyelets on a shoe. In the device 200, the end elements are intentionally of a greater diameter than the thread (body portion), to maintain the device from in the bandage into which it is inserted, until the bandage is removed, panel-by-panel, therefrom, as described hereinbelow.)

Regarding the dimensions of the device 200, the device 200 may be sized and shaped to extend through the several panels of a zig-zag folded bandage, such as the bandage 100 illustrated in FIG. 1A. In other words, the device is long enough (sufficiently long) to extend through the thickness (“T”, see FIG. 1A) of a folded-up bandage, and is thin enough (sufficiently thin) to pierce through the material of the web of the bandage.

Some exemplary dimensions may be:
the length “L” of the body portion 202 may be approximately 25-35 mm, such as 30 mm
the thickness/diameter “D1” of the body portion 202 may be approximately 1 mm (the thickness/diameter “D1” of the body portion 202 is adapted to fit through the web material, for example, the weave)
the thickness/diameter “D2” of the end elements 204a/204b may be approximately 2.5 mm (the thickness/diameter “D2” of the end elements 204a/204b is adapted to retain the web panels folded, unless a user pulls one or more web panels, in which case the web material (generally elastic material) stretches and end elements 204a/204b can fit through the web releasing one or more web panels from the device).
the thickness “T” of the folded-up bandage may be approximately 30 mm (L˜T)

FIG. 2A illustrates the device 200 inserted through a folded-up bandage 250 which may be substantially identical to the bandage 100 of FIG. 1A. The bandage 250 (compare 100) has a web 252 (compare 102), six folds 254a-254f (compare 104a-104f) forming seven panels 252a-252g (compare 102a-102g), a dressing 258 (compare 108) and a pressure enhancement member 260 (compare 110).

The top end element 204a is adapted to be disposed exterior the panel 252a, which may be considered to be a “first” of the folded-up panels, and this is the panel which incorporates the dressing 258. The bottom end element 204b is disposed exterior the panel 252g, which may be considered to be a “last” of the folded-up panels.

Note that the length “L” of the body portion 202 of the device 200 is approximately equal to the thickness “T” of the folded-up bandage 250, both being approximately 30 mm. The length “L” of the body portion 202 of the device 200 may be slightly (such as up to a few millimeters) greater than the thickness “T” of the folded-up bandage 250. Or, the length “L” of the body portion 202 of the device 200 may be slightly (such as up to a few millimeters) less than the thickness “T” of the folded-up bandage 250, in which case the folded-up bandage 250 could be compressed before inserting the device 200 through it. As mentioned above, the thickness (or diameter) of the device 200 is such that it can be inserted (pierce) through the web material of the bandage. (It is believed that inserting the device 200 through the dressing which is adapted to contact the wound is not recommendable.)

The overall idea is that the device “captures” the folded-up bandage 250, retaining it in its folded configuration, and inhibiting it from unfurling (unfolding), of its own accord. In use, however, a user can unfold the bandage 250 without removing the device 200 (with the device still inserted into the folded-up bandage 250).

For example, as the user pulls on an end panel 252a of the bandage 250, which is the topmost “retained” panel, the end panel 252a can slip over the top end element 204b, and will become a “released” panel, and the next panel 252b becomes the topmost “retained” panel. This is illustrated, for example, in FIG. 2B, where the arrow 270 indicates the user pulling the end panel 252a away from the rest of the panels 252b-252g. The end panel 252a thereby slips over the top end element 204a of the device 200, and becomes “released”, rather than “retained”. End panel 252a may then be placed on a wound, such that the dressing 258 contacts the wound surface. End panel 252a is kept on the wound, while the user may continue with the un-folding process.

As noted above, the end panel 252a (compare 102a) with the dressing 258 may be slightly longer than the rest of the panels 252b-g, and constitutes is an “additional” web portion 256 (compare 106), which typically extends about 2 cm from the dressing 108 and which may be grasped by a user when applying the bandage 250 (compare 100) on a wound (not shown).

After placing the free panel 252a on the wound (such that dressing 258 contacts the wound), panels 252b-g may be pulled down in a way that the topmost retained panel 252b, will be released This is illustrated, for example, in FIG. 2C, where the arrow 272 indicates the user pulling down panels 252c-252g. The panel 252b then becomes a “released” panel, and the next panel 252c becomes the topmost “retained” panel, and so forth, sequentially releasing panels 252c-252f, until the user stops pulling and releasing panels, or all but the last panel 252g are released. This can be done in a “controlled release” manner, wherein the user releases the panels (or the folds) one by one as per his/her need (“Stop and Go”). In other words, a type of “interference fit” is formed between the device 200 and the web material of the bandage 250. As panels are pulled, they can slide over the end element 204a of the device 200, panel-by-panel, freeing themselves therefrom. (Actually, the last panel to be “released” would be the next to the last panel 262f, since after the next to the last panel 252f is released, there are no more panels left for the last panel 252g to be released from. However, after all the releasable panels 252a-252f are released, the device 200 can be removed from the last panel 252g.

FIGS. 2B and 2C illustrate the bandage unfurling (unfolding), with the device 200, in response to the user pulling, thereby releasing panels which were retained by the device 200, in conjunction with the use applying the bandage 250 to a wound (not shown). Compare FIG. 1B, which has no such device to restrain the bandage from “self-unfurling” (unfurling of its own accord).

The angle between the panels 252a and 252b (in FIGS. 2B and 2C), and between the panels 252b and 252c (in FIG. 2C) would likely be greater than as shown, rather than angles of approximately 20 degrees, more like an angle of 180 degrees (in line with each other).

FIG. 2D illustrates a fold-retaining device 220 which is substantially identical to the fold-retaining device 200 of FIG. 2A, with the following difference. The device 220 (compare 200) has a body portion 222 (compare 202) with two opposite (“top” and “bottom”) ends 222a and 222b (compare 202a and 202b). Whereas, in FIG. 2A, the top and bottom end elements 204a and 204b are shown as being the same as one another, in FIG. 2B, the bottom end element 224b is shown being different than the top end element 224a. The top end element 222a is ball-shaped and has a diameter “D2”, as is the top end element 202a. But, the bottom end element 222b is flat, and may be disc shaped, having a diameter “D3” which is greater than “D2”. The device 220 may be made from the same materials as the device 200, as discussed above.

FIG. 2E illustrates a fold-retaining device 240 which is similar to the fold-retaining device 200 of FIG. 2A, in that it has a body portion 242 (compare 202) with two opposite (“top” and “bottom”) ends 242a and 242b (compare 202a and 202b). However, whereas in FIG. 2A, there are only top and bottom end elements 204a and 204b (and both are shown as being ball-shaped and having the same diameter as one another), the fold-retaining device 240 has a number of ball-shaped intermediate elements 244b-244g of increased diameter disposed along the body portion 242, between the top end element 244a and the bottom end element 244h.

The body portion 242 has a diameter “D1”. The top element 244a has a diameter “Da” which is greater than “D1”. The bottom element 244h has a diameter “Dh” which is greater than “D1”. The intermediate elements 242b-242g each have a respective diameter “Db”−“Dg” which may be greater than the diameter “D1” of the body portion 242. The intermediate elements 242b-242g may have various and assorted diameters, for example, alternating between intermediate elements 244b, 244d and 244f having a smaller diameter, and intermediate elements 244c, 244e and 244g having a larger diameter, generally as illustrated. Or, for example, the diameters of the intermediate elements 244b-244g may increase from a smallest diameter for the element 244b to a largest diameter for the element 244g. The diameter of any of the intermediate elements 244b-244g can be smaller or larger than (or the same as) the diameter of the top and bottom elements 244a and 244h. Many variations are possible. The general idea is to have “bumps” (the intermediate elements) between each layer of material in the folded (or rolled) bandage. An alternative would be to have, instead of some or all of the bumps, to have depressions (regions of diameter less than “D1”) at various places along the body portion 242.

It has thus been shown that a number of elements (top, bottom, intermediate) can be disposed at the ends of and along the body portion (202, 222, 242) in various configurations to aid in retaining the bandage in its folded (or rolled) configuration.

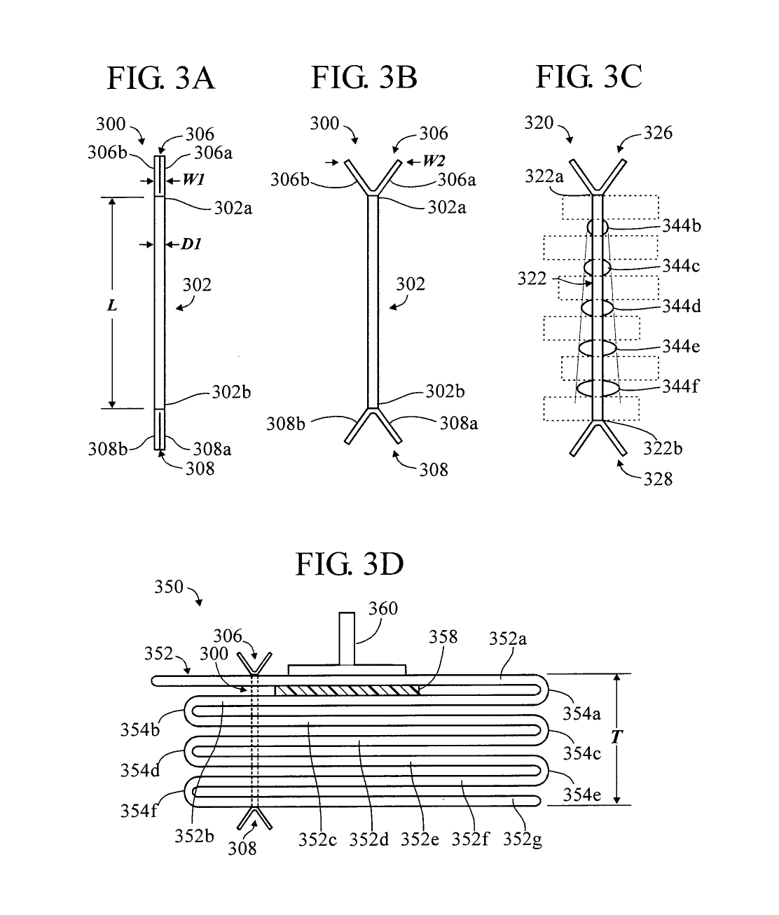

FIGS. 3A and 3B illustrates a fold-retaining device 300 which is similar in some respects to the fold-retaining device 200 of FIG. 2A. The device 300 (compare 200) is an elongated member, such as in the form of a pin. The device 300 has a body portion 302 with two opposite ends 302a and 302b. The body portion 302 may be round in cross-section. The body portion 302 has a length “L” and a thickness or diameter (if round in cross-section) “D1”. For purposes of this description, the end 302a is a “top” end, and the end 302b is a “bottom end”.

The body portion 302 may be formed of a structural plastic material. Or, it may be formed as a thread, such as cotton.

End elements 306 and 308 are noticeably different than the end elements 204a and 204b of FIG. 2. Whereas in FIG. 2, the end elements 204a and 204b are ball-shaped, in the embodiment of FIGS. 3A and 3B, the end elements 306 and 308 are Y-shaped, or “bifurcated”. For purposes of this description, the end element 306 is a “top” end element, and the end element 308 is a “bottom” end element.

The end element 306 may be formed having two elongated fingers 306a and 306b, the fingers extending longitudinally from (in the same direction as) the elongated body portion 302, generally, as longitudinal extensions thereof. Each finger 306a and 306b may have a thickness which is approximately half of the thickness/diameter D1 of the body portion 302. For example, each finger may have a thickness of 0.5 mm. The fingers 306a and 306b may have a length (in the vertical direction, as drawn) of approximately 3 mm.

The end element 308 may be similarly formed having two elongated fingers 308a and 308b, the fingers extending longitudinally in the same direction as the elongated body portion 302, generally, as longitudinal extensions thereof. Each finger 308a and 308b may have a thickness which is approximately half of the thickness/diameter D1 of the body portion 302. For example, each finger may have a thickness of 0.5 mm. The fingers 308a and 308b may have a length (in the vertical direction, as drawn) of approximately 3 mm.

In FIG. 3A, the end elements 306 and 308 are shown collapsed, having a width dimension “W1” which is substantially equal to the diameter D1 of the body portion 302. In FIG. 3B, the end elements 306 and 308 are shown spread-out (or simply “spread”, or not collapsed), having an overall width dimension “W2” which is greater than the diameter “D1” of the body portion 302. In FIG. 3B, the end elements are shown spread out. In practice, the bottom end element 308 may need not be collapsed, and may always be spread-out.

Spread out, as shown in FIG. 3B, would be the normal, “relaxed” position for the fingers 306a/306b and 308a/308b. Formed of a resilient material, the fingers, such as the fingers 306a and 306b of the top end element 306 can be resiliently urged (compressed) together by an insertion tool (not shown) and, after being inserted through a web of a folded-up bandage, and eliminating the compression, the fingers 306a and 306b will naturally return to their spread-out position.

The end elements 306 and 308 may be formed integrally with the body portion 302 (such as, for example, in the case of shirt tag holders), or separately therefrom and appended (mounted) thereto. The end elements 306 and 308 may be formed, for example, of plastic by injection molding, the fingers of the end elements 306 and 308 in their spread position.

Regarding the dimensions of the device 300, the device 300 may be sized to extend through a zig-zag folded bandage, such as the bandage 100 illustrated in FIG. IA. In other words, the device is long enough (sufficiently long) to extend through the thickness (“T”, see FIG. 1A) of a folded-up bandage, and is thin enough (sufficiently thin) to pierce through the material of the web of the bandage.

Some exemplary dimensions may be:
the length “L” of the body portion 302 may be approximately 25-35 mm, such as 30 mm.
the thickness/diameter “D1” of the body portion 202 may be approximately 1 mm.
the width W1 of the end elements 306/308, when collapsed, may be approximately 1 mm.
the width W2 of the end elements 306/308, when spread, may be approximately 2.5 mm

In use, the situation when using the fold-retaining device 300 is essentially identical to that when using the fold-retaining device 200.

FIG. 3C (compare FIG. 2A) illustrates the device 300 (compare 200) inserted through a folded-up bandage 350 which may be substantially identical to the bandage 100 of FIG. 1A (or the bandage 250 of FIG. 2A). The bandage 350 (compare 100, 250) has a web 352 (compare 102, 252), six folds 354a-354f (compare 104a-104f, 254a-254f) forming seven panels 352a-352g (compare 102a-102g, 252a-252g), a dressing 358 (compare 108, 258) and a pressure enhancement member 360 (compare 110, 260).

The top end element 306 (compare 204a) is adapted to be disposed exterior the panel 302a (compare 252a), which may be considered to be a “first” of the folded-up panels, and this is the panel which incorporates the dressing 358. The bottom end element 308 (compare 204b) is disposed exterior the panel 302g (compare 252g), which may be considered to be a “last” of the folded-up panels.

Note that the length “L” of the body portion 302 of the device 300 is approximately equal to the thickness “T” of the folded-up bandage 350, both being approximately 30 mm. The length “L” of the body portion 302 of the device 300 may be slightly (such as up to a few millimeters) greater than the thickness “T” of the folded-up bandage 350. Or, the length “L” of the body portion 302 of the device 300 may be slightly (such as up to a few millimeters) less than the thickness “T” of the folded-up bandage 350, in which case the folded-up bandage 350 could be compressed before inserting the device 300 through it. However, this kind of “compression retaining” may work better with the device 200 than with the device 300.

As described above, in connection with the device 220, the top end element 224a can be different than the bottom end element 224b. It should be understood that any of the top and bottom end elements described herein can be “mixed and matched” to arrive at different configurations.

The overall idea is the same as with the device 200—namely, that the device 300 “captures” the folded-up bandage 350, not allowing it to unfurl (unfold), of its own accord. In use, however, a user can unfold the bandage 350.

For example, as the user pulls on an end panel 352a of the bandage 350, the end panel 352a can slip over the top end element 306. Subsequent pulling on the bandage will subsequently release subsequent panels 352b-352g. Compare FIGS. 2B and 2C above.

FIG. 3D illustrates an embodiment of a device 320 similar in some respects to the device 300 of FIGS. 3A and 3B, in that the device comprises a body portion 322 (compare 302) with two opposite ends 322a and 322b (compare 302a and 302b). And the end elements 326 and 328 (compare 306 and 308) are Y-shaped, or “bifurcated”. For purposes of this description, the end element 326 is a “top” end element, and the end element 328 is a “bottom” end element.

The device 320 is also provided with in a manner similar to that of the device 240 of FIG. 2E. A number of intermediate elements 344b-344f (compare 244b-244g) of increased diameter are disposed along the body portion 322, between the top end element 326 and the bottom end element 328. (For illustrative clarity, fewer intermediate elements are shown on the device 320 than were shown on the device 240.)

The intermediate elements 344b-344f each have a respective diameter which may be greater than the diameter (“D1”) of the body portion 322. For example, as mentioned above, the intermediate elements 344b-344f may have different diameters, increasing from a smallest diameter for the element 344b to a largest diameter for the element 344f. The dashed-line boxes between the various top, bottom and intermediate elements are schematically illustrative of layers of web material being releasably retained between adjacent elements. Faint guidelines are included to show how the diameters of the intermediate elements 344b-344f may increase in a “tapered” manner.

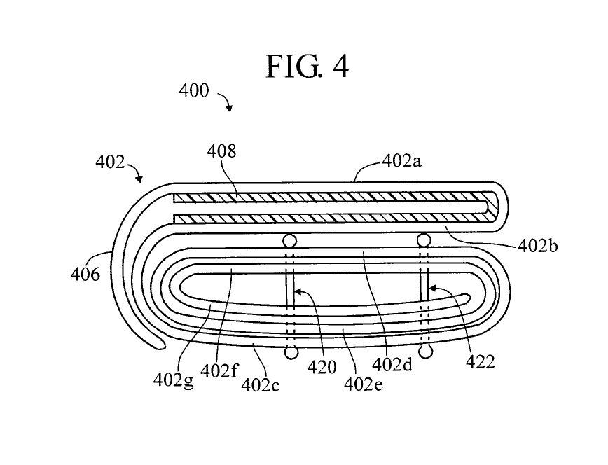

FIG. 4 illustrates another embodiment of the invention. The bandage 400 (comparable to 100) has a web 402 (comparable to 102) comprising seven web portions 402a-402g (comparable to panels 102a-102g), of course seven is only a an example and any number of web portions can be present. A dressing 408 (comparable to 108) is disposed on one side of the web. The portions 402a and 402b are folded, and the dressing 408 is also folded and is disposed between these folded portions 402a and 402b. The end portion 402a of the web extends beyond the dressing 408 to form an “additional” web portion 406 (comparable to 106).

In this embodiment, the web is not folded in a zig zag manner. Rather, the web portions 402c-402g may be rolled up, in a flattened spiral manner. (This should also be considered to be a type of “folding”.) One or more (two shown) retaining devices 420 and 422 (compare 200) are shown, extending through and releasably retaining the rolled up portions 402c-402g. Notice that each of these devices 420 and 422 have top and bottom end elements (not labeled, comparable to 204a, 204b) (Generally, the portions 402a and 402b, with the dressing 408, may not be pierced by the device(s).) The bandage 400 is shown loosely folded/rolled, in a kind of “exploded view”, for illustrative clarity.

As before, the overall idea is that the retaining device(s) “captures” the rolled-up bandage 400, retaining it in its folded (including rolled) configuration, and inhibiting it from unfurling (unfolding, unrolling), of its own accord. In use, however, a user can unfold (or unroll) the bandage 250 without removing the device(s) 200 (with the device still inserted into the folded-up bandage 250).

Alternatively, instead of (or in addition to) using a fold-retaining device or more specifically, a “string” (as in the previous embodiments), a heated needle (or a metal wire or any other element) may be inserted through the folded bandage, which may cause the fibers of the web (generally, elastic synthetic fibers) that are in a direct contact with the heated needle to melt and stick to one or more adjacent folds (web sections or panels) in the area of the needle path. The needle can then be removed, and the web may be maintained folded by the melted track of the needle path. Unfolding can then be done gradually (such as panel-by-panel, layer-by-layer), as with the other embodiments. There is thus provided, according to some embodiments, a bandage comprising an elongated web adapted to be folded upon itself (or rolled or folded in a zig zag manner), the web, when being in a folded configuration, comprises a plurality of folds or web sections or panels abutting one another, wherein the folded configuration is adapted to be releasably retained by one or more areas in which two adjacent folds (web sections or panels) are melted together. The “melting together” of the one or more areas may be accomplished as discussed above by the insertion of a heated needle (or a metal wire or any other element).

According to some embodiments there is provided a method for releasably retaining an elongated web of a bandage in a folded configuration, the method includes: folding an elongated web of a bandage such that the web includes a plurality of folds (web sections or panels) abutting one another); and inserting a fold-retaining device through two or more of the plurality of panels thereby releasably retaining the web in a folded configuration.

According to some embodiments there is also provided a method for releasably retaining an elongated web of a bandage in a folded configuration, the method includes:

folding an elongated web of a bandage such that the web includes a plurality of folds (web sections or panels) abutting one another; and inserting a heated element (such as a needle or a wire) through two or more of the plurality of folds, wherein fibers of one fold (generally, elastic synthetic fibers) that are in a direct contact with the heated element are melted and stick to fibers of one or more adjacent folds in the area of the needle path, thereby releasably retaining the web in a folded configuration.

The fold-retaining devices 200, 220, 240, 300 described hereinabove may be inserted into the web of a folded-up bandage using a tool (referred to as a “gun”) such as is used for clothing tags.

In the description and claims of the application, each of the words “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. It is intended that the scope of the invention be limited only by the claims and that the claims be interpreted to include all such variations and combinations.

1-55. (canceled)

56. A bandage comprising:
an elongated web adapted to be folded upon itself, the web, when being in a folded configuration, comprises a plurality of panels abutting one another; and
a fold-retaining device comprising an elongated body portion, a top end element and a bottom end element opposing to the top end element, wherein the device is adapted to extend through two or more of the plurality of panels and releasably retain the web in a folded configuration.

57. The bandage of claim 56, wherein the fold-retaining device is adapted to allow a user to gradually unfold the web without removing the device.

58. The bandage of claim 56, wherein the fold-retaining device is adapted to allow a user to unfold one or more panels by slipping the one or more panels, panel-by-panel, over the top end element of the fold-retaining device, while maintaining other panels folded.

59. The bandage of claim 56, wherein the device is sufficiently long to extend through the folded web, and is sufficiently thin to pierce through a material of the web of the bandage.

60. The bandage of claim 56, wherein a length (L) of the body portion of the fold-retaining device is approximately equal to a thickness (T) of the folded web.

61. The bandage of claim 56, wherein a length (L) of the body portion of the fold-retaining device is slightly greater than a thickness (T) of the folded web.

62. The bandage of claim 56, wherein a length (L) of the body portion of the fold-retaining device is slightly less than a thickness (T) of the folded web.

63. The bandage of claim 56, wherein the web is zigzag folded.

64. The bandage of claim 56, further comprising a dressing adapted to cover a wound, the dressing is disposed on a surface of a selected one of the panels.

65. The bandage of claim 64, further comprising a pressure enhancement member disposed o n an opposite surface of the selected one of the panels.

66. The bandage of claim 56, wherein at least one of the top end element and the bottom end element has a thickness (D2), which is greater than a thickness (D1) of the body portion.

67. The bandage of claim 56, wherein the body portion is essentially round in cross-section, and the thickness (D1) is a diameter.

68. The bandage of claim 56, wherein the body portion comprises one or more materials selected from the group consisting of polymers, synthetic polymers, silicon, and cotton.

69. The bandage of claim 56, wherein the body portion comprises a thread, which comprises a textile material comprising natural fibers, synthetic fibers or a combination thereof.

70. The bandage of claim 56, the main body portion comprises a thread and the end elements are formed of a rigid material.

71. The bandage of claim 56, wherein the top end element is adapted to be disposed exterior a first of the panels, and the bottom end element is adapted to be disposed exterior a last of the folded panels.

72. The bandage of claim 56, wherein at least one of the top and bottom end elements are essentially ball-shaped.

73. The bandage of claim 56, wherein at least one of the top and bottom end elements is Y-shaped.

74. The bandage of claim 73, wherein:
the Y-shaped end element, when collapsed, has a width dimension (W1) which is substantially equal to a thickness (D1) of the body portion; and
the Y-shaped end element, when spread, has a width dimension (W2) which is greater than the thickness (D1) of the body portion.

75. The bandage of claim 56, wherein the top and bottom end elements are substantially the same as one another.

76. The bandage of claim 56, wherein the top and bottom end elements are different from one another.

77. The bandage of claim 56, wherein at least one of the top and bottom end elements are integrally formed with the body portion.

78. The bandage of claim 56, wherein at least one of the top and bottom end elements are formed separately from the body portion, and are mounted thereto.

79. The bandage of claim 56, wherein at least one of the top and bottom end elements comprises two or more elongated fingers extending in a spread position from the body portion, wherein the two or more elongated fingers are adapted to collapse when one or more panels are being pulled to unfold the web.

80. A fold-retaining device comprising:
an elongated body portion, a top end element and a bottom end element opposing to the top end element, wherein the device is adapted to extend through two or more panels of an elongated folded web of a bandage, and releasably retain the web in a folded configuration.

81. Method of releasably retaining an elongated web of a bandage in a folded configuration, the method comprising:
folding an elongated web of a bandage to form a plurality of panels abutting one another; and
inserting a fold-retaining device through two or more of the plurality of panels such that the fold-retaining device is releasably retaining the web in a folded configuration.

82. Method of releasably retaining an elongated web of a bandage in a folded configuration, the method comprising:
folding an elongated web of a bandage to form a plurality of panels abutting one another; and
inserting a heated element through two or more of the plurality of folds, wherein fibers of one fold that are in a direct contact with the heated element are melted and stick to fibers of one or more adjacent folds in the area of the path of the heated element, thereby releasably retaining the web in a folded configuration.

83. The method of claim 82, further comprising compressing the folded bandage, prior to inserting the heated element.
